(19) United States
(12) Patent Application Publication
(10) Pub. No.: US 2022/0118132 A1
(43) Pub. Date: Apr. 21, 2022

(54) System and Method of Sanitizing a Package Left at a Doorstep

(71) Applicants: Allan Mark Klepfisz, Tel Aviv Yafo (IL); Lance Ford, Pound Ridge, NY (US); Tomas Varga, Bratislava (SK)

(72) Inventors: Allan Mark Klepfisz, Tel Aviv Yafo (IL); Lance Ford, Pound Ridge, NY (US); Tomas Varga, Bratislava (SK)

(21) Appl. No.: 17/364,872

(22) Filed: Jun. 30, 2021

Related U.S. Application Data
(60) Provisional application No. 63/018,305, filed on Apr. 30, 2020.

(51) Int. Cl. A61L 2/10; A61L 2/24; A61L 2/28; G07C 9/00; G07C 9/21

(52) U.S. Cl. CPC A61L 2/10; A61L 2/24; A61L 2/28; G07C 9/00182; G07C 2009/00253; G07C 9/21; A61L 2202/11; A61L 2202/14; A61L 2202/23; G07C 9/00896

(57) ABSTRACT

A system and method of sanitizing a package left at a doorstep safely reduces exposure to pathogens with packages. The method includes a computerized doorbell, at least one motion detector, at least one ultraviolet device, and a user (PC) personal computing device. The method begins by scanning a surrounding area of the doorway with the motion detector in order to identify at least one physical package. A delivery notification for the physical package is relayed from the motion detector, through the computerized doorbell, and to the user PC device. The delivery notification is displayed for the physical package with the motion detector. The surrounding area is further scanned with the motion detector in order to identify a human presence within the surrounding area. Ultraviolet radiation is emitted onto the surrounding area for a specified time period with the ultraviolet device, if the physical package is identified within the surrounding area.

The current application claims a priority to the U.S. provisional patent application Ser. No. 63/018,305 filed on Apr. 30, 2020. The U.S. provisional patent application 63/018,305 is revived within the two-month period for unintentional abandonment by Jun. 30, 2021.

### FIELD OF THE INVENTION

The present invention generally relates to package deliveries. More specifically, the present invention sanitizes a package left at a doorstep.

### BACKGROUND OF THE INVENTION

The delivery of packages is one of the essential aspects of modern-day life. It allows one to receive items such as food or other essentials without having to leave one's housing. One aspect of having packages delivered is that there is a lower chance of being exposed to pathogens than there is going to a local store. However, the chance of being exposed to pathogens while having packages delivered is still significant. This is especially problematic when certain pathogens have been known to live on packaging materials for hours. Therefore, there is a need to further reduce one's exposure to pathogens when having packages delivered.

The present invention is a method for handling packages and delivery safely by significantly reducing exposure to pathogens. The present invention accomplishes this by minimalizing contact between an individual and the courier. The present invention allows one to be notified of a package and pay the courier without having to be in direct contact with said courier. The present invention additionally makes use of ultraviolet light to kill off any pathogens on the packages that have been delivered to one's housing. The present invention additionally has a locked storage chamber for storing payment to the courier. The courier can then receive a passcode to unlock said storage chamber to receive their payment without having to be in direct contact with the package receiver. The present invention also makes use of sensors to detect the presence of a package and the respective courier. This allows an individual to keep track of how much time has passed since the courier has touched the package. The tracking of time can be used to determine if any potential pathogens still remain on the delivered packages.

### BRIEF DESCRIPTION OF THE DRAWINGS

FIG. 1 is a schematic view of the system of the present invention.

FIG. 2 is a flowchart illustrating the overall process for a method of the present invention.

FIG. 3 is a flowchart illustrating the subprocess of activating two-way communication between a user personal computing (PC) device and a computerized doorbell.

FIG. 4 is a flowchart illustrating the subprocess of initiating and terminating a timer with the ultraviolet device.

FIG. 5 is a flowchart illustrating the subprocess of displaying a sanitation-complete notification if ultraviolet device has been activated for a specified time period.

FIG. 6 is a flowchart illustrating the subprocess of periodically displaying a delivery notification until a physical package has been picked up.

FIG. 7 is a flowchart illustrating the subprocess of storing and opening an electronic lockbox if a deliverer PC device has received a current access code from the user PC device.

FIG. 8 is a flowchart illustrating the subprocess of replacing the current access code with a new access code that was manually inputted into the user PC device.

FIG. 9 is a flowchart illustrating the subprocess of replacing the current access code with a new access code that automatically created by the user PC device.

### DETAIL DESCRIPTIONS OF THE INVENTION

All illustrations of the drawings are for the purpose of describing selected versions of the present invention and are not intended to limit the scope of the present invention.

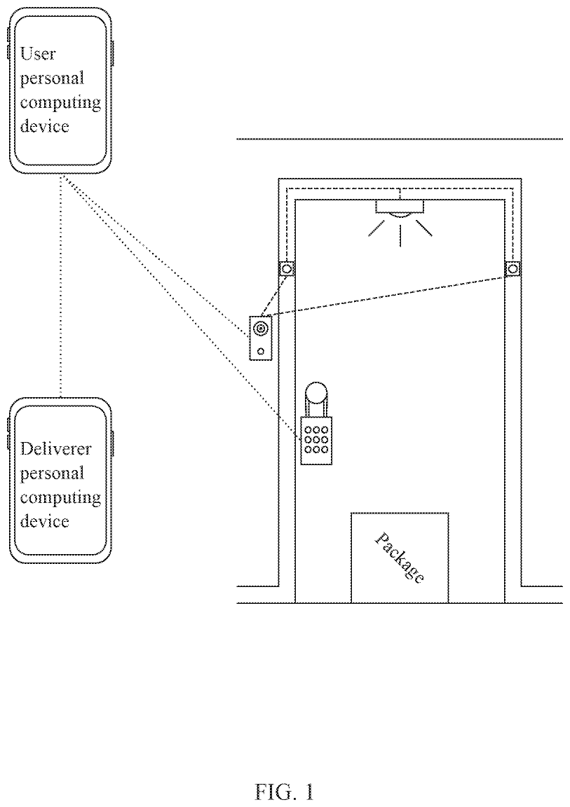

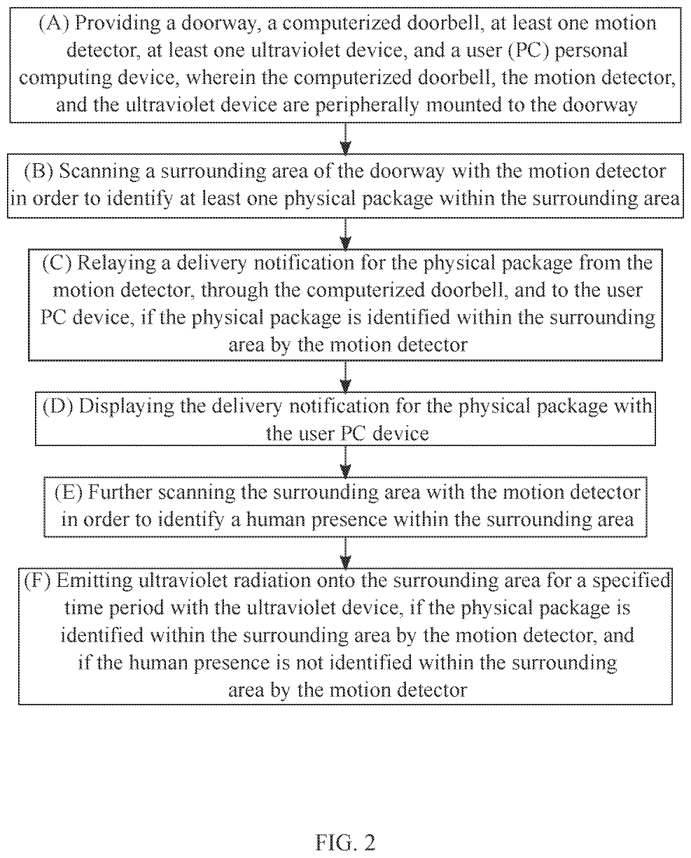

The present invention is a system and method of sanitizing a package left at a doorstep. The present invention significantly reduces exposure to pathogens. More specifically, the present invention reduces the amount of contact between a recipient with a courier. The reduction of contact ensures that the exposure of pathogens is further mitigated. Thus, the physical system used to implement the method for the present invention includes a doorway, a computerized doorbell, at least one motion detector, at least one ultraviolet device, and a user personal computing (PC) device (Step A), as seen in FIG. 1 and FIG. 2. The doorway is an entranceway such as a door frame that physically defines a path into and out of a designated area. As packages are left at a designated doorway for a recipient, the computerized doorbell, the motion detector, and the ultraviolet device are peripherally mounted to the doorway. The computerized doorbell allows a courier to alert the recipient of his or her presence, as well as provide a means for communication between the recipient and the courier without being in direct contact with each other. The at least one motion detector determines the presence of a package or an individual near the doorway. The at least one ultraviolet device provides a source of ultraviolet light that kills off pathogens of a package. More specifically, the at least one ultraviolet device is preferably an ultraviolet germicidal lamp. The user PC device remotely manages the corresponding computerized doorbell, the at least one motion detector, and the at least one ultraviolet device. The user PC device is preferably a smartphone but can also be, but is not limited to, a desktop, a tablet PC, a smartwatch, and a laptop.

The overall process for the method of the present invention includes the following steps that are implemented with the doorway, the computerized doorbell, the at least one motion detector, the at least one ultraviolet device, and the user PC device. As seen in FIG. 2, the method begins by scanning a surrounding area of the doorway with the motion detector in order to identify at least one physical package within the surrounding area (Step B), thereby determining if the ultraviolet device needs to be activated. The physical package may be a box containing a shipped item, a bag of groceries, a bag of takeout food, and so on. Upon the detection of a package, a delivery notification for the physical package is relayed with the motion detector, through the computerized doorbell, and to the user PC device, if the physical package is identified within the surrounding area by the motion detector (Step C). The delivery notification informs the recipient of the presence of a package and differentiates the presence of a package and an individual. In order for the recipient to view the delivery notification, the delivery notification for the physical package is displayed with the user PC device (Step D). The safety of the courier and other individuals is maintained as the surrounding area is further scanned with the motion detector in order to identify a human presence with the surrounding area (Step E) due to the harmful effects of concentrated amounts of ultraviolet light for humans. The physical package is effectively sanitized before the recipient comes into contact with the package as ultraviolet radiation is emitted onto the surrounding area for a specified time period with the ultraviolet device, if the physical package is identified within the surrounding area by the motion detector, and if the human presence is not identified within the surrounding area by the motion detector (Step F).

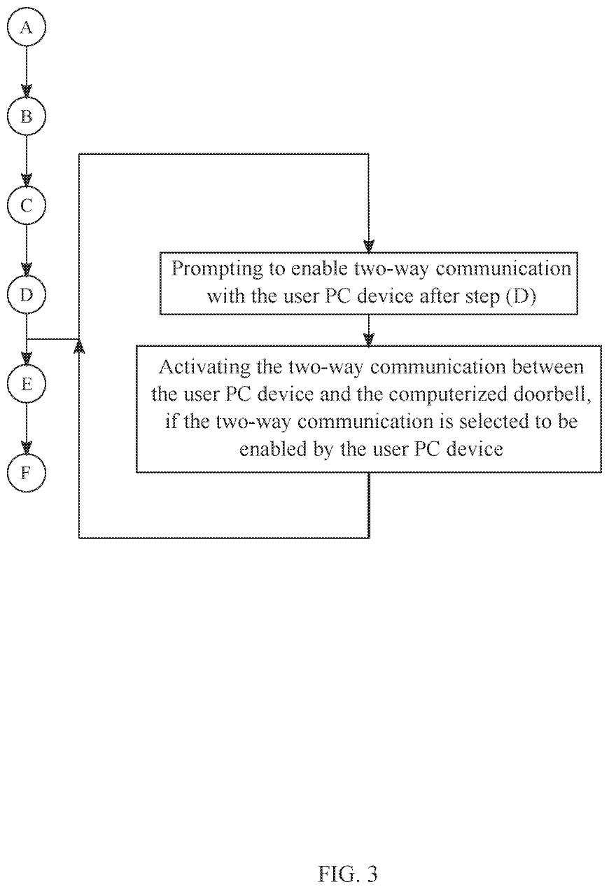

In order for a recipient of the physical package to communicate with a courier, the recipient is prompted to enable two-way communication with the user PC device after Step D, as seen in FIG. 3. In the preferred embodiment of the present invention, the computerized doorbell further includes a camera, a speaker, a microphone, and a wireless communication device in order to facilitate communication between computerized doorbell and the user PC device. The two-way communication between the user PC device and the computerized doorbell is activated, if the two-way communication is selected to be enabled by the user PC device. It is understood that the computerized doorbell may be activated by a ringer button as well. The ringer button delivers relays a ring notification to the user PC device. The ring notification is preferably a message that is displayed with the user PC device and an audio alert that is emitted with the user PC device.

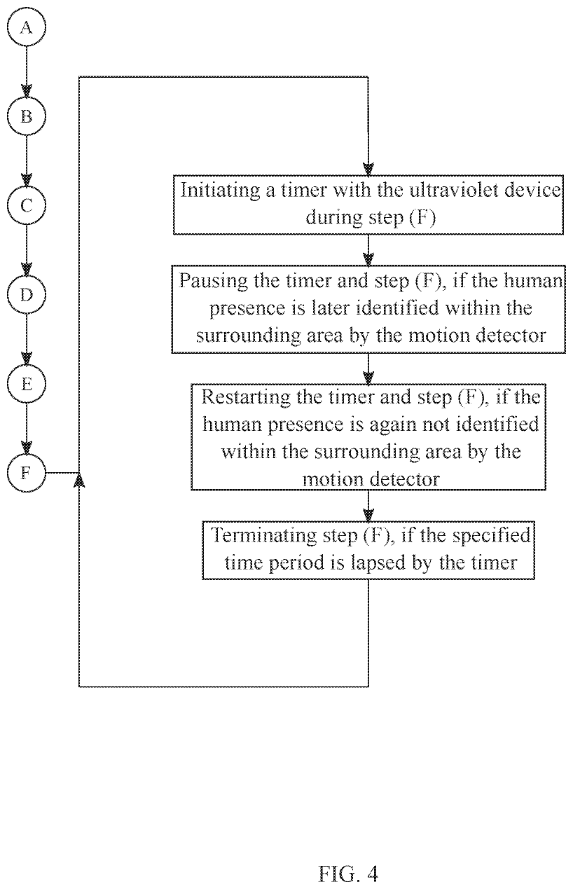

In order to effectively sanitize a physical package, a timer is initiated with the ultraviolet device during Step F, as seen in FIG. 4. The timer designates a specific amount of time necessary to apply ultraviolet light onto the package for complete sanitization. The timer and Step F are paused, if the human presence is later identified within the surrounding area by the motion detector. This ensures the package receives the necessary exposure to ultraviolet light while preventing the courier or another individual from being exposed to concentrated amounts of ultraviolet light. The timer and Step F are restarted, if the human presence is again not identified within the surrounding area by the motion detector. More specifically, the timer and Step F continues on from the point the timer has paused. Once the package has been completely sanitized, Step F is terminated, if the specified time period is lapsed by the timer.

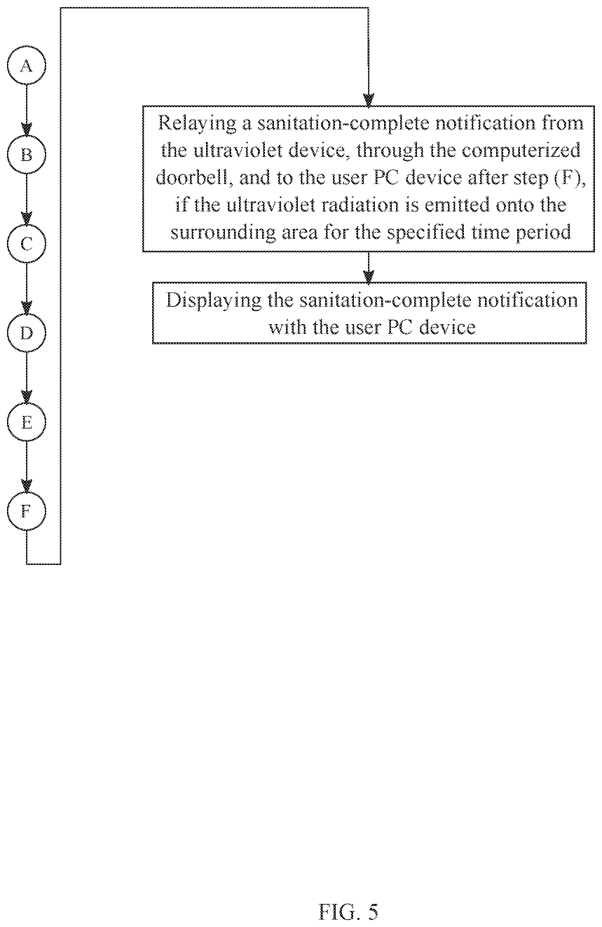

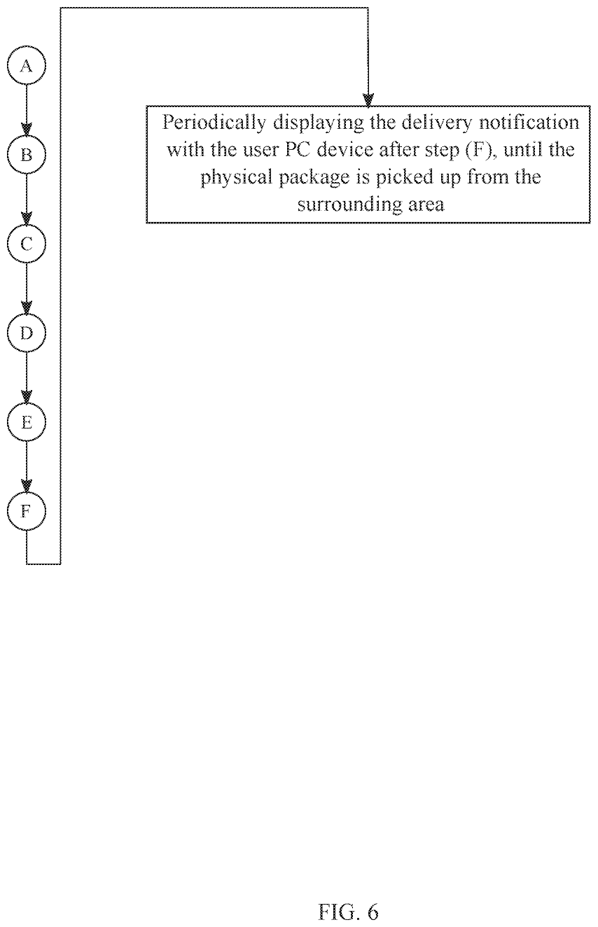

In order for the recipient to know when the physical package is ready for pickup at the doorway, a sanitation-complete notification is relayed from the ultraviolet device, through the computerized doorbell, and to the user PC device after Step F, if the ultraviolet radiation is emitted onto the surrounding area for the specified time period, as seen in FIG. 5. The sanitation-complete notification alerts the user that the physical package has been effectively and thoroughly sanitized. The sanitation-complete notification is displayed with the user PC device, alerting the recipient with the user PC device. Furthermore, the delivery notification is periodically displayed with the user PC device after Step F, until the physical package is picked up from the surrounding area, so that the physical package is not contaminated between sanitization and pickup, seen in FIG. 6.

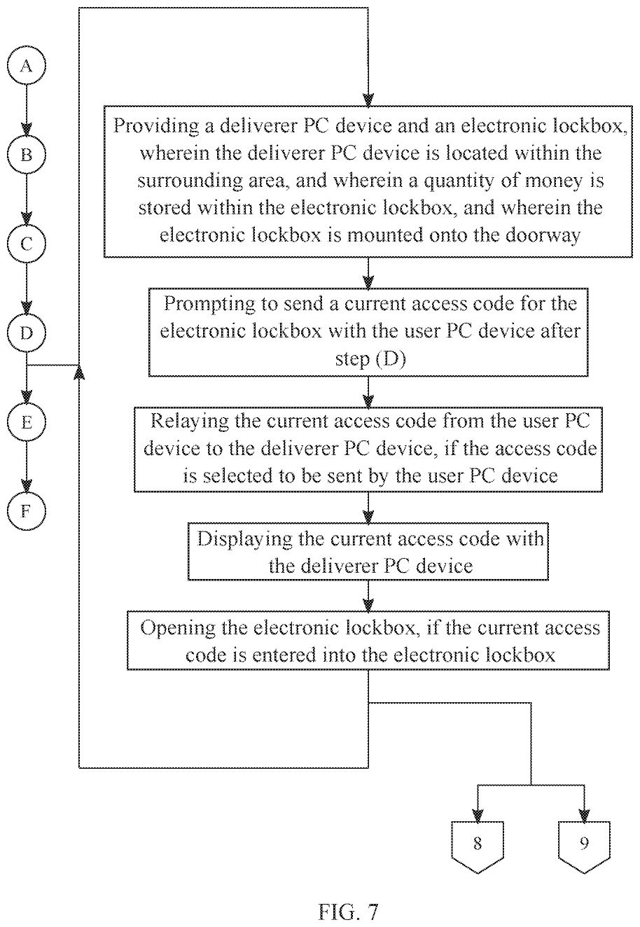

The system of the present invention further includes a deliverer PC device and an electronic lockbox, as seen in FIG. 1 and FIG. 7. Similar with the user PC device, the deliverer PC device is preferably a mobile computing device (e.g., a smartphone) that is able to communicate with the user PC device. The deliverer PC device may also communicate with a remote server so that the courier may register the delivery of the physical package and securely communicate with a corresponding user PC device. The electronic lockbox is preferably a door lockbox that is secured with a doorknob or doorhandle. The recipient may securely tip or pay a courier as a quantity of money is stored within the electronic lockbox, and the electronic lockbox is mounted onto the doorway, thereby preventing theft. In order to grant access for the courier, the recipient is prompted to send a current access code for the electronic lockbox with the user PC device after Step D so that the courier may receive the tip or payment only upon the completion of delivery. The current access code is preferably a series of numbers that may be entered into the electronic lockbox with a keypad of the lockbox. In order for the courier to receive the current access code, the current access code is relayed from the user PC device to the deliverer PC device, if the access code is selected to be sent by the user PC device. The current access code is displayed with the deliverer PC device, allowing the courier to view the current access code with the deliverer PC device. The courier may physically retrieve the quantity of money as the electronic lockbox is opened, if the current access code is entered into the electronic lockbox.

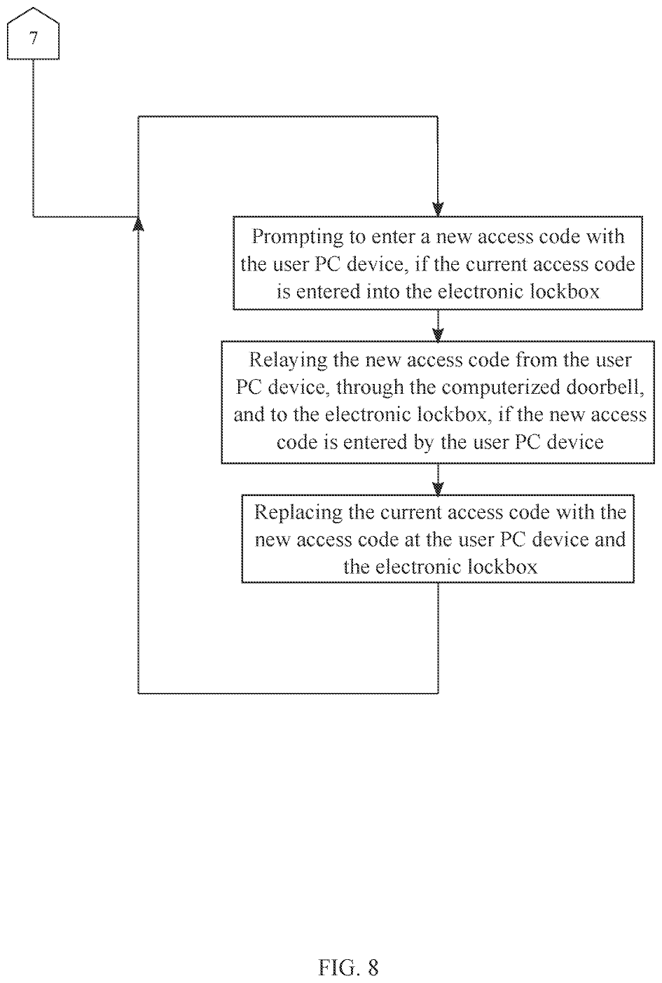

In order for a recipient to manage each access code between couriers, the recipient is prompted to enter a new access code with the user PC device, if the current access code is entered into the electronic lockbox, as seen in FIG. 8. The new access code is preferably a series of numbers that is different than the series of numbers of the current access code and is chosen by the recipient. This prevents the previous courier from being able to access the quantity of money within the electronic lockbox that is designated for another courier. The new access code is defined as the new access code is relayed from the user PC device, through the computerized doorbell, and to the electronic lockbox, if the new access code is entered by the user PC device. The current access code is replaced with the new access code at the user PC device and the electronic lockbox, thereby providing each courier with a unique access code.

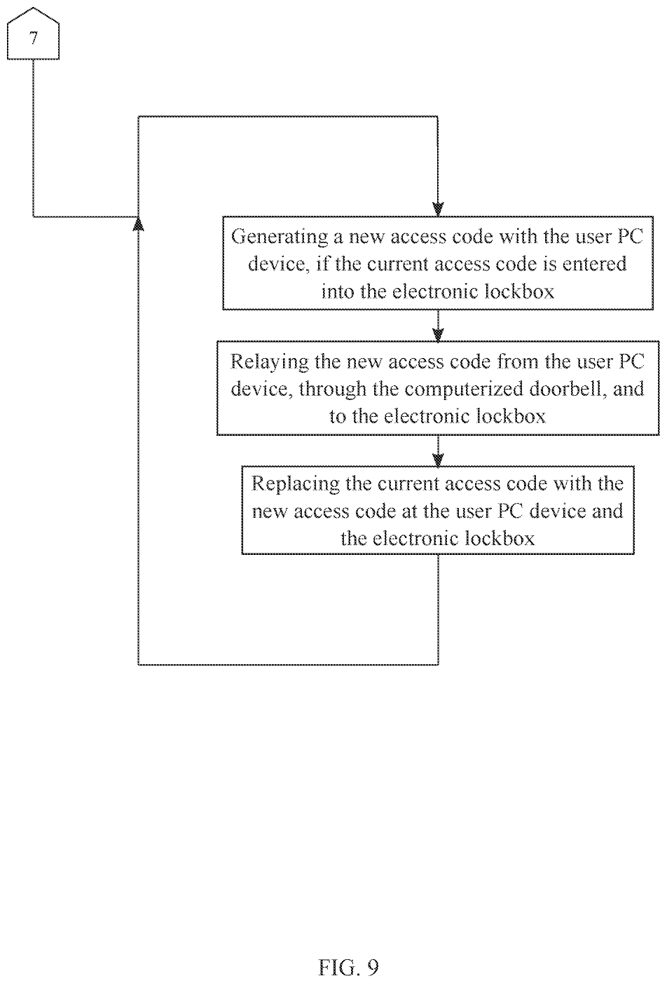

Each access code may be automatically changed between each courier as a new access code is generated with the user PC device, if the current access code is entered into the electronic lockbox so that the recipient does not have to manually enter a new access code between each delivery, as seen in FIG. 9. Similarly, the new access code is defined as the new access code is relayed from the user PC device, through the computerized doorbell, and to the electronic lockbox, if the new access code is entered by the user PC device. The current access code is automatically replaced as the current access code is replaced with the new access code at the user PC device and the electronic lockbox.

Although the invention has been explained in relation to its preferred embodiment, it is to be understood that many other possible modifications and variations can be made without departing from the spirit and scope of the invention as hereinafter claimed.

What is claimed is:

1. A method of sanitizing a package left at a doorstep, the method comprising the steps of:
(A) providing a doorway, a computerized doorbell, at least one motion detector, at least one ultraviolet device, and a user (PC) personal computing device, wherein the computerized doorbell, the motion detector, and the ultraviolet device are peripherally mounted to the doorway;
(B) scanning a surrounding area of the doorway with the motion detector in order to identify at least one physical package within the surrounding area;
(C) relaying a delivery notification for the physical package from the motion detector, through the computerized doorbell, and to the user PC device, if the physical package is identified within the surrounding area by the motion detector;
(D) displaying the delivery notification for the physical package with the user PC device;
(E) further scanning the surrounding area with the motion detector in order to identify a human presence within the surrounding area; and,
(F) emitting ultraviolet radiation onto the surrounding area for a specified time period with the ultraviolet device, if the physical package is identified within the surrounding area by the motion detector, and if the human presence is not identified within the surrounding area by the motion detector.

2. The method of sanitizing a package left at a doorstep, the method as claimed in claim 1 comprising the steps of:
prompting to enable two-way communication with the user PC device after step (D); and,
activating the two-way communication between the user PC device and the computerized doorbell, if the two-way communication is selected to be enabled by the user PC device.

3. The method of sanitizing a package left at a doorstep, the method as claimed in claim 1 comprising the steps of:
initiating a timer with the ultraviolet device during step (F);
pausing the timer and step (F), if the human presence is later identified within the surrounding area by the motion detector;
restarting the timer and step (F), if the human presence is again not identified within the surrounding area by the motion detector; and,
terminating step (F), if the specified time period is lapsed by the timer.

4. The method of sanitizing a package left at a doorstep, the method as claimed in claim 1 comprising the steps of:
relaying a sanitation-complete notification from the ultraviolet device, through the computerized doorbell, and to the user PC device after step (F), if the ultraviolet radiation is emitted onto the surrounding area for the specified time period; and,
displaying the sanitation-complete notification with the user PC device.

5. The method of sanitizing a package left at a doorstep, the method as claimed in claim 1 comprising the step of:
periodically displaying the delivery notification with the user PC device after step (F), until the physical package is picked up from the surrounding area.

6. The method of sanitizing a package left at a doorstep, the method as claimed in claim 1 comprising the steps of:
providing a deliverer PC device and an electronic lockbox, wherein the deliverer PC device is located within the surrounding area, and wherein a quantity of money is stored within the electronic lockbox, and wherein the electronic lockbox is mounted onto the doorway;
prompting to send a current access code for the electronic lockbox with the user PC device after step (D);
relaying the current access code from the user PC device to the deliverer PC device, if the access code is selected to be sent by the user PC device;
displaying the current access code with the deliverer PC device; and,
opening the electronic lockbox, if the current access code is entered into the electronic lockbox.

7. The method of sanitizing a package left at a doorstep, the method as claimed in claim 6 comprising the steps of:
prompting to enter a new access code with the user PC device, if the current access code is entered into the electronic lockbox;
relaying the new access code from the user PC device, through the computerized doorbell, and to the electronic lockbox, if the new access code is entered by the user PC device; and,
replacing the current access code with the new access code at the user PC device and the electronic lockbox.

8. The method of sanitizing a package left at a doorstep, the method as claimed in claim 6 comprising the step of:
generating a new access code with the user PC device, if the current access code is entered into the electronic lockbox;
relaying the new access code from the user PC device, through the computerized doorbell, and to the electronic lockbox; and,
replacing the current access code with the new access code at the user PC device and the electronic lockbox.
